(19) United States
(12) Patent Application Publication
(10) Pub. No.: US 2019/0083808 A1
(43) Pub. Date: Mar. 21, 2019

(54) APPARATUS AND METHOD FOR EMITTING LIGHT TO A BODY OF A USER

(71) Applicant: Jessica Iverson, Jersey City, NJ (US)

(72) Inventor: Jessica Iverson, Jersey City, NJ (US)

(21) Appl. No.: 15/710,785

(22) Filed: Sep. 20, 2017

(51) Int. Cl. A61N 5/06

(52) U.S. Cl. CPC A61N 5/0613; A61N 2005/0635; A61N 2005/0664; A61N 2005/0659; A61N 2005/0662; A61N 2005/0651

(57) ABSTRACT

An apparatus is provided. The apparatus includes a flexible wrap and a plurality of lights arranged on the wrap. The wrap is configured to wrap around a body of a user such that the lights face selected areas of the body. The apparatus is configured to cause the lights to emit light at a first predetermined wavelength to the selected areas of the body that is sufficient to perforate one or more fat cells at the selected areas of the body to drain the content within the one or more fat cells when power is supplied to the lights. The apparatus is configured to cause the lights to emit light at a second predetermined wavelength to the selected areas of the body at the same time as the lights emit light at the first predetermined wavelength when power is supplied to the lights.

### FIELD

This application relates to an apparatus and method for emitting light to a body of a user. In particular, this apparatus includes a flexible wrap that is configured to wrap around the body of the user and lights on the wrap that are operated to emit light at a predetermined wavelength to areas of the body to increase lipolysis of the fat cells at the areas of the body.

### BACKGROUND

Known light emitting diode (LED) devices may emit infra-red and near infra-red light of a specified wavelength that can provide therapeutic effects to persons exposed to the light under certain conditions. Other light devices may include the use of low-level laser (LLT) therapy, which also involves exposing body tissues of persons to red and near infrared light. Exposure to the above type of light under certain conditions can alleviate various effects that sun exposure, gravity, pollution, and harsh soaps and chemicals have on the skin. The devices may take the form of a flexible pad having an array of LEDs or other lights on the pad that can emit the infra-red light on a user's body. For example, these devices may come in the form of a flexible pad that can be secured to the back, leg, shoulder, elbow or other parts of a user's body to temporarily reduce pain from arthritis, trauma, and strains.

Light devices may benefit from improvements.

### SUMMARY

In one embodiment, an apparatus is provided. The apparatus includes a flexible wrap and a plurality of lights arranged on the wrap. The wrap is configured to wrap around a body of a user such that the lights face selected areas of the body. The apparatus is configured to cause the lights to emit light at a first predetermined wavelength to the selected areas of the body that is sufficient to perforate one or more fat cells at the selected areas of the body to drain the content within the one or more fat cells when power is supplied to the lights. The apparatus is configured to cause the lights to emit light at a second predetermined wavelength to the selected areas of the body at the same time as the lights emit light at the first predetermined wavelength when power is supplied to the lights.

In another embodiment, a method is provided. The method includes wrapping a wrap assembly around a body of a user such that a plurality of lights on the wrap face selected areas of the body, and supplying power to the wrap to cause the lights to emit light at a first predetermined wavelength to the selected areas of the body that is sufficient to perforate one or more fat cells at the selected areas of the body to drain the content within the one or more fat cells and simultaneously emit light at a second predetermined wavelength to the selected areas of the body to increase circulation at the selected areas of the body.

Other embodiments of the disclosed apparatus and method emitting light to a body of a user will become apparent from the following detailed description, the accompanying drawings and the appended claims.

### BRIEF DESCRIPTION OF THE DRAWINGS

FIG. 1 is perspective view of the apparatus according to an embodiment of the invention;

FIG. 2 is a perspective view of the apparatus of FIG. 1 showing the wrap wrapped around the body of a user;

FIG. 3 is a front view of the wrap of the apparatus of FIG. 1 with the sheet removed for illustrative purposes;

FIG. 4 is a block diagram of the components of the apparatus of FIG. 1;

FIG. 5 is a block diagram of the first and second integrated circuits connected to the LEDs of the apparatus of FIG. 1;

FIG. 6 is a top view of chips according another embodiment of the apparatus of FIG. 1;

FIG. 7 is a top perspective view of a chip of FIG. 6; and

FIG. 8 is a flow diagram of a method for emitting light to a body of a user using the apparatus of FIG. 1.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

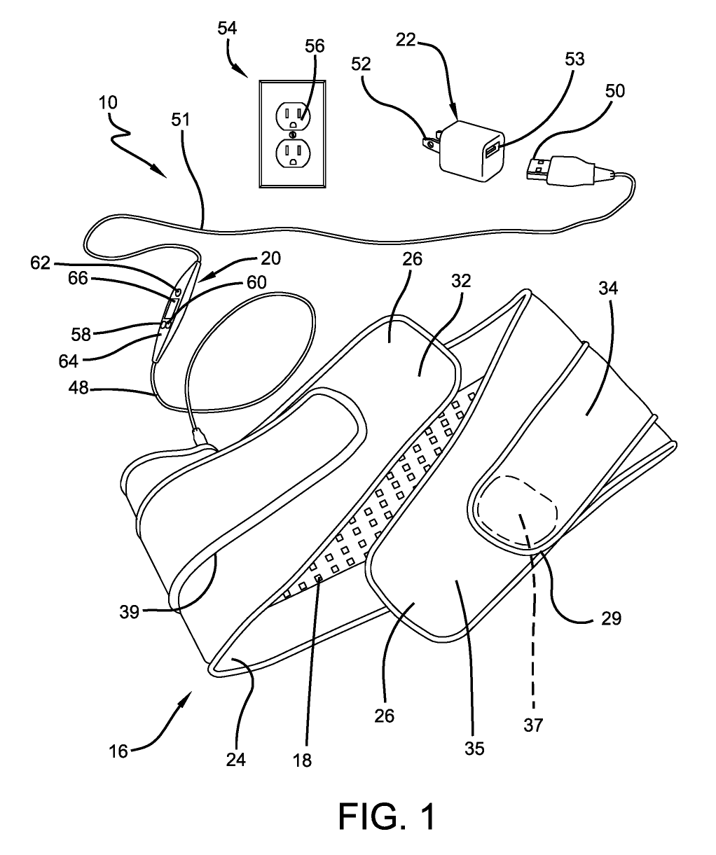

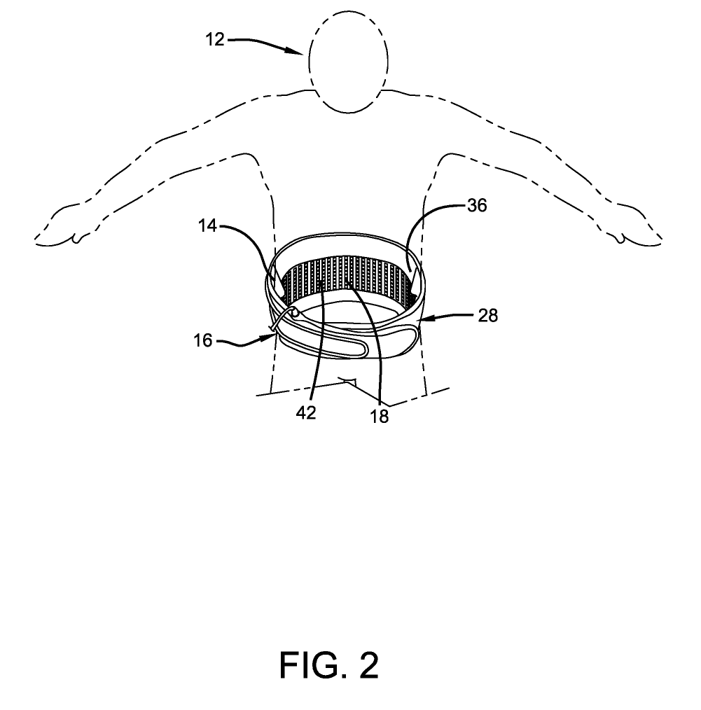

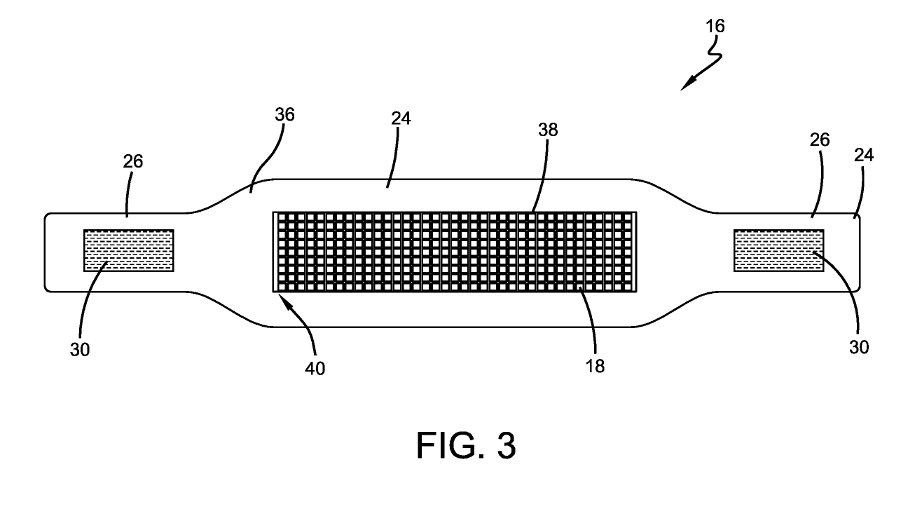

FIGS. 1-6 illustrate an embodiment of an apparatus 10 and method for emitting light to a body 14 of a user 12. Referring to FIG. 1, the apparatus 10 comprises a flexible elongated wrap 16, light emitting diode (LED) lights 18, a switch controller 20, and a power adapter 22. The wrap 16 may be a silica fiber cloth or other suitable main body belt. The wrap 16 is elongated and may be 116 cm in length. The wrap 16 includes a central body portion 24 and opposite arms 26 that extend axially from the body portion 24. The wrap 16 includes a hook and pile fastener 28 (FIGS. 2 and 3) such as Velcro® thereon to detachably attach the arms 26 or other parts of the wrap 16 together when the wrap is wrap around an object. In particular, a first lineal fabric strip 30 comprising small hooks is sewn or otherwise adhered to one or each arm 26 at the front side 36 of the wrap 16 and a second lineal fabric strip 32 (FIG. 1) comprising smaller loops is sewn or otherwise adhered to the rear side 35 of the wrap 16. When the strips 30, 32 are pressed together, the hooks catch in the loops and the two arms 26 fasten or bind temporarily together. A looped fabric strip defining a handle 34 (FIG. 1) is attached to the rear side 35 of the wrap 16 and extends from one arm 26 along the body portion 24 of wrap 16 to the other arm 26. The looped fabric strip 34 may be detachably attached to the arms 26 at the rear side 35 of the wrap 16 by a hook and pile fastener 29 (FIGS. 1 and 2) such as Velcro®. In particular, referring to FIG. 1, a third lineal fabric strip 37 comprising small hooks is sewn or otherwise adhered to the front side 39 of the handle 34 at one or each axial end of the handle 34. When the third and second strips 37, 32 are pressed together, the hooks catch in the loops and the handle 34 and wrap 16 are fasten or bind temporarily together. Alternatively, the looped fabric strip 34 may be sewn or otherwise secured at its axial ends to their respective arms 26.

As illustrated in FIG. 3, the body portion 24 has a rectangular recess 38 at the front side 36 of the wrap 16. The surface 40 of the recess 38 may comprise a uniform color such as black. The surface may alternatively comprise a reflective color surface such as a mirrored surface. Other alternative embodiments may have the surface 40 be comprises of a non-mirrored uniform color or non-uniformly colored. The apparatus 10 includes an array of the lights 18 mounted on the surface 40 of the recess 38 or otherwise secured to the wrap 16 at the location of the recess 38. The lights 18 may be covered by a flexible sheet 42 (FIG. 2) made of plastic or other suitable material. The sheet 42 may be transparent and extend over the recess 38 to allow the light emitted by the lights 18 to shine through. The lights 18 may comprise infra-red LEDs. The apparatus 10 may comprise 300 LEDs arranged as 30 columns of LEDs with each column including 10 rows of LEDs. Alternative embodiments may include additional rows and/or columns of LEDs. Alternative embodiments may also include less LEDs. Additional embodiments may employ near infra-red LEDs in addition to, or in place of, any of the infra-red LEDs.

One embodiment uses the specific configuration and number of LEDs in order to provide the level of infra-red light necessary to perforate one or more fat cells at the selected areas of the body to drain the content within the one or more fat cells to enhance fat loss and detoxification of the user's body. This level of infra-red light also increases the lipolysis of the fat cells at the areas of a user's body 14 facing the LEDs when the wrap 16 is wrapped around the body 14. The current embodiment also uses the specific configuration and number of LEDs in order to provide the level of infra-red light necessary to reduce pain and inflammation at the areas of the body. This level of infra-red light also boosts circulation of the body and penetrates deeply into the body. This level of infra-red light also stimulates production of nitric oxide in the blood which opens up the capillaries of the user's body, thus increasing the blood flow. This level of infra-red light also creates a metabolic effect to burn calories. Alternative embodiments may include non infra-red LEDs. The power for the apparatus is between 5 watts and 20 watts during operation of the apparatus 10. In one example, power for the apparatus is 20 watts during operation of the apparatus. The heat generated by the LEDs may cause the temperature of the space outwardly adjacent the LEDs to be 104 degrees. The recess 38 defines the treatment area of the body of the user. In one example, the treatment area is 18 inches by 5 inches. In another example, the treatment area may be 19.7 inches by 4.3 inches (11 cm by 50 cm).

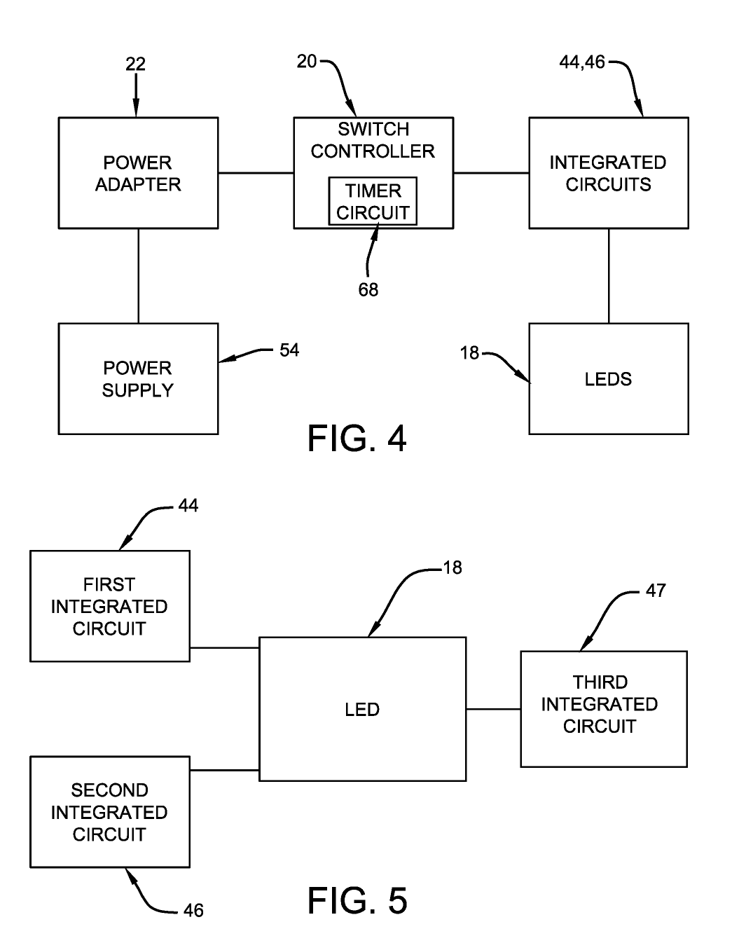

As illustrated in FIGS. 4 and 5, each of the LEDs may be electrically coupled to first and second circuits 44, 46. The first and second circuits may be first and second integrated circuits or chips 44, 46. The first and second integrated circuits 44, 46 may be mounted on a printed circuit board or other suitable platform. The printed circuit board may be flexible. The output of the switch controller 20 is electrically coupled to the first and second integrated circuits 44, 46 via an electric cord 48 (FIG. 1). The input of the switch controller 20 is electrically coupled to the power adaptor 22 via an electric cord 51. In particular, the free end of the electric cord comprises a USB connector 50 that may be plug into a USB port 53 of the power adapter 22 as illustrated in FIG. 1. Alternatively, the power adapter 22 may not have a USB port and the end of the electric cord may not have the USB connector and instead may be electrically coupled to the power adapter 22. The power adapter 22 includes plugs 52 that may be plugged into slots 56 of an electrical outlet 54 or other power source that supplies between 100-240 VAC at 50/60 herz. For example, the power adapter 22 may be plugged into an electrical outlet that supplies 120 VAC at 60 herz. The power ranking of the apparatus 10 may be 1, 2, 3, 4, or 5. Alternatively, instead of the electrical outlet 54 and power adapter 22, the power source 54 may be a battery that supplies a 12 volt DC.

The first integrated circuit 44 is configured to cause the LEDs 18 to emit infrared radiation at a first predetermined wavelength to the selected areas of the body that is sufficient to perforate one or more fat cells at the selected areas of the body to drain the content within the one or more fat cells. This emitted infrared radiation at the first predetermined wavelength also increases the lipolysis of the fat cells at the areas of a user's body 14 facing the LEDs when the wrap 16 is wrapped around the body 14. The first predetermined wavelength may be between 630 nm and 635 nm. In one exemplary embodiment, the wavelength may be 635 nm.

The second integrated circuit 46 is configured to cause the LEDs 18 to emit infrared radiation at a second predetermined wavelength that reduces pain and inflammation at the areas of the body facing the LEDs when the wrap 16 is wrapped around the body 14. This emitted infrared radiation at the second predetermined wavelength boosts circulation of the body and penetrates deeply into the body. This emitted infrared radiation at the second predetermined wavelength stimulates production of nitric oxide in the blood which opens up the capillaries of the user's body, thus increasing the blood flow. This emitted infrared radiation at the second predetermined wavelength also creates a metabolic effect to burn calories. The second predetermined wavelength may be between 830 nm and 880 nm. In one exemplary embodiment, the second predetermined wavelength may be 850 nm. The emitted radiation may be FAR-Infrared in 850 nm wavelength.

The apparatus may optionally include a third circuit 47. The third circuit may be a third integrated circuit 47 as seen in FIG. 5. Each of the LEDs may be electrically coupled to third integrated circuit 47. The third integrated circuit 47 may be mounted on a printed circuit board or other suitable platform. The printed circuit board may be flexible. The output of the switch controller 20 is electrically coupled to the third integrated circuit 476 via the electric cord 48. The third integrated circuit 47 is configured to cause the LEDs 18 to emit infrared radiation at a third predetermined wavelength that increase collagen formation in the selected areas of the body at the areas of the body facing the LEDs when the wrap 16 is wrapped around the body 14, which helps speed up the healing of injuries such as cuts. This emitted infrared radiation at the third predetermined wavelength also boosts cellular energy by stimulating Adenosine triphosphate (ATP). This emitted infrared radiation at the third predetermined wavelength also penetrates up to ¾ of an inch into the skin and helps to flatten the skin and smooth skin wrinkles. In one exemplary embodiment, the second predetermined wavelength may be 660 nm. The emitted radiation may be near infrared in the 660 nm wavelength. Infrared radiation at this wavelength may also be visible to the user. First, second, and third integrated circuits may be separate integrated circuits or chips, or circuits that are provided on a single chip.

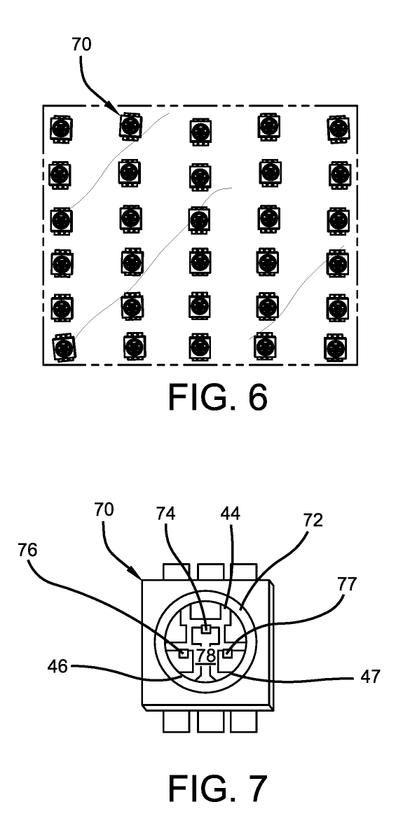

FIGS. 6 and 7 show exemplary chips 70 for the LEDs 18. As illustrated in FIG. 7, each chip 70 comprises the first, second, and third circuits 44, 46, 47. A mounting recess 72 is formed in the top of the chip and securely receives an LED 18. First, second, and third output ports 74, 76, 77 are provided on the bottom side 78 of the recess 72. The output of the switch controller 20 is electrically coupled to the chips 70 via the electric cord 48. The output signals of the first, second, and third circuits 44, 46, 47 are outputted through their corresponding first, second, and third output ports 74, 76, 77. The output signals cause the LED 18 to emit radiation at the area of the LED 18 adjacent or otherwise corresponding to respective out ports. This enables the LED 18 to emit radiation in three wavelengths (e.g. 635 nm, 660 nm, and 850 nm) at the same time.

Alternatively or in addition, the first and/or second and/or third and/or additional integrated circuits or circuits may be configured to cause the LEDs to emit light at wavelengths between 307 nm and 347 nm (purple light spectrum), 620 nm and 638 nm (red light spectrum), 415 nm and 475 nm (blue light spectrum), and 575 nm and 620 nm (yellow light spectrum).

The switch controller 20 may have three settings or modes that enable a user to select which function or treatment that the wrap 16 performs. The switch controller 20 may be a variety of types. For example, as illustrated in FIG. 1, the switch controller 20 may be membrane touch switch that has first, second, and third push buttons 58, 60, 62 that are each pressed by a finger of the user 12 to select the desired mode of operation of the apparatus 16. The switch controller 20 may include a plastic housing 64 that houses the electronic components of the switch controller 20. The switch controller 20 may include a display 66 to indicate the mode of operation and other information such as the time. Alternatively, the switch controller 20 may include a touch screen for the user to select the desired mode of operation. In another example, the switch controller may be configured to enable a user to select the desired mode of operation by a cell phone or a remote controlled device. The first push button 58 may be pressed or otherwise activated to place the apparatus 16 in the weight loss mode in which the LEDs 18 first predetermined wavelength to the selected areas of the body that is sufficient to perforate one or more fat cells at the selected areas of the body to drain the content within the one or more fat cells and also increases the lipolysis of the fat cells at the areas of a user's body 14 facing the LEDs when the wrap 16 is wrapped around the body 14.

The second push button 60 may be pressed or otherwise activated to place the apparatus in a pain therapy mode in which the LEDs 18 emit infrared radiation at a wavelength that reduces pain and inflammation at the areas of the body facing the LEDs when the wrap 16 is wrapped around the body 14. The third button 62 may be pressed or otherwise activated to place the apparatus in the visible light mode. In the visible light mode, the LEDs 18 emit infrared radiation at a third predetermined wavelength that increase collagen formation in the selected areas of the body at the areas of the body facing the LEDs when the wrap 16 is wrapped around the body 14. Also, in the visible mode, non infra-red LEDs emit light at a wave length of approximately 660 nm. Non infra-red light of this wavelength is useful because (i) light of this wavelength is within the visible spectrum, so the user knows the device is powered on and working; and (ii) the wavelength of light may possess therapeutic qualities for skin. It should be noted that any non-infrared LEDs that produce visible light may be utilized in place of the 660 nm LEDs for informing the user that the unit is currently active and operational. A fourth button may be added to the switch controller 20 or another suitable way may be used to activate a timer that allows operation of the wrap 16 for a predetermined time. The time may be 10, 20, 30, 40, 50 or 60 minutes. The switch controller may have another push button or other suitable way to place the switch controller in a more aggressive pain therapy mode. Alternatively, the fourth push button may be used for a different setting or be eliminated from the switch controller. It is further contemplated that in other variations LEDs may be used that produce infrared light in any part of the infrared spectrum.

Alternatively or in addition, the switch controller 20 may be configured to have one push button that will cycle through the three modes and set the time of operation. For example, the user may press the first push button 58 of the switch controller 20 once to place the switch controller 20 in the weight loss mode. The user then may then press the first push button 58 a second time to place the switch controller 20 in the pain therapy mode. A user may then press the first push button 58 a third time to place the switch controller in the visible light mode. A user may then press the first push button 58 a fourth time and hold it down for a number of seconds to set the wrap 16 to operate for a first predetermine time. If the user does not want the wrap 16 to operate at the first predetermined time, a user may then press the first push button 58 a fifth time and hold it down for a number of seconds to set the wrap 16 to operate for a second predetermine time. Further times of pressing of the first push button 58 and holding it down for a number of seconds may set the wrap 16 to operate for other selected predetermine times. When at the portion of the cycle that sets the predetermine time of operation of the wrap 16, pressing the first push button 58 and not holding it down for a number of seconds will not select the corresponding predetermine time of operation of the wrap, but enable the user to select the next predetermined time. After the user presses the push button 58 the last time to select a predetermine time of operation of the wrap 16, or cycle past the last predetermine time of operation, the cycle begins again to enable the user to select the three modes of operation beginning with the weight loss mode.

The switch controller may also be configured in combination with the chips of FIGS. 6 and 7 to enable operation of the weight loss, pain therapy, and visible light modes at the same time such that one or more or all of the LEDs emit radiation of the three different wavelengths at the same time. Alternatively or in addition, the switch controller or may also be configured in combination with the chips 70 of FIGS. 6 and 7 to enable operation of a combination of two of the modes of operation at the same time such that one or more or all of the LEDs emit radiation of the selected two different wavelengths at the same time. For example, the first push button may be pressed a fourth time to have all three modes operate at the same time, a fifth time to have just the weight loss and pain therapy mode operate at the same time, a sixth time to have just the pain therapy mode and visible light mode operate at the same time, and a seventh mode to have just the weight loss and visible light mode operate at the same time. A user may press the first push button an eighth time to start the cycle portion to select the predetermined time of operation of the wrap 16.

The switch controller 20 may be configured to enable the user 12 to select via a push button or other suitable selector a first group of LEDs 18 to emit light at the first predetermined wavelength (e.g. 635 nm) to the selected areas of the body facing the first group of LEDs that is sufficient to perforate one or more fat cells at the selected areas of the body to drain the content within the one or more fat cells when the wrap is placed at that position by a user 12. The switch controller 20 may be configured to enable the user 12 to select via a push button or other suitable selector a second group of LEDs 18 to emit light at the second predetermined wavelength (e.g. 850 nm) that reduces pain and inflammation at the selected areas of the body 14 facing the second group of LEDs when the wrap 16 is placed at that position by a user 12. The switch controller 20 may be configured to enable the user 12 to select via a push button or other suitable selector a third group of LEDs 18 to emit light at the third predetermined wavelength (e.g. 660 nm). In one exemplary embodiment, the user may select 150 LEDs as the first group and 150 LEDs as the second group. The first, second, and third groups of LEDs may all operate to emit infrared radiation at the same time or just one or combination of two of the first, second, and third groups may operate to emit infrared radiation.

Alternatively or additionally, the apparatus may be configured to enable the wrap 16 to operate the first group of LEDs or a predetermined number of LEDs in the first group at one or more of the modes of operation for a predetermined time and/or enable the wrap 16 to operate the second group of LEDs or a predetermined number of LEDs in the second group at one or more of the modes of operation for a predetermined time and/or to enable the wrap 16 to operate the third group of LEDs or a predetermined number of LEDs in the third group at one or more of the modes of operation for a predetermined time. Alternatively or additionally, the apparatus may be configured to enable the wrap 16 to operate at one or more modes of operation for a predetermined time and then operate at a different mode or combination of modes for a predetermined time without the user having to select the different mode or combination of modes of operation.

The switch controller 20 may include a timer circuit 68 (FIG. 4) that modulates the infrared light emitted by the LEDs 18 such that LEDs 18 turn off the light for one second for every four seconds. This increases the user's body cells ability to respond to the infrared radiation. Alternatively, the timer circuit 68 may be a separate element located outside of the switch controller 20.

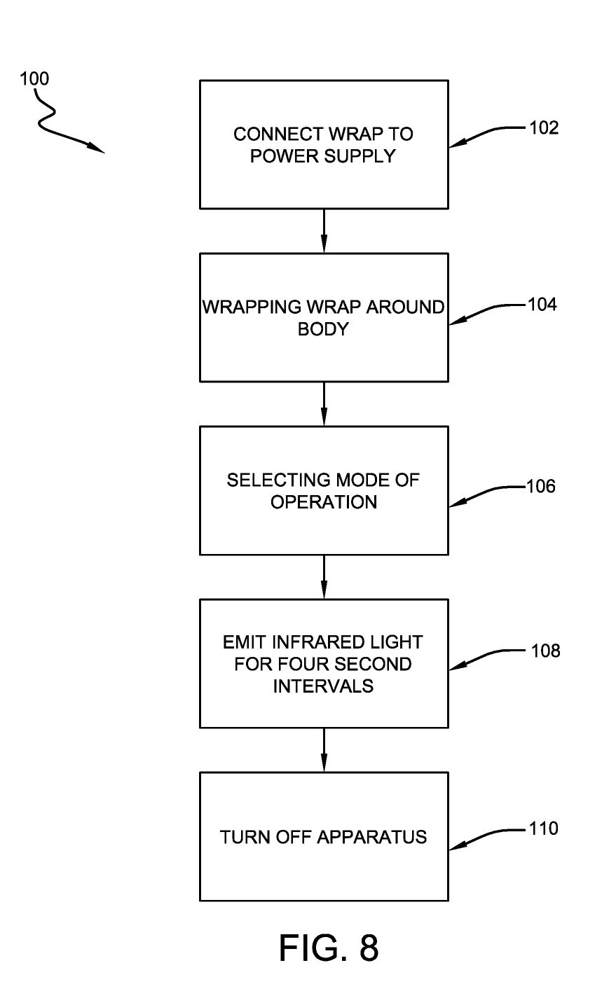

FIG. 8 illustrates an exemplary method 100 for using and operating the apparatus. In step, 102, the power adapter 22 is plugged into the electrical outlet 54 and the USB connector 50 is plugged into the USB port 53 of the power adapter 22. In step 104, the user 12 grasps the handle 34 and wraps the wrap 16 around the body 14 of the user 12 such that front side 36 of the wrap 16 faces the body of the user as shown in FIG. 2. This step includes pressing the lineal fabric strips 30, 32 together to detachably attach the arms 26 of the wrap together. In step 106, the user selects the mode of operation. For example, the user 12 may press the first push button 58 of the switch controller 20 to place and operate the apparatus 16 in the weight loss mode. The time that the LEDs 18 operate to emit the infrared radiation may be also selected by the user 12. When the apparatus 16 is operated in the weight loss mode, power is supplied from the electrical outlet 54 into the power adaptor 22, where it is converted into a 12 volt DC. The 12 volt DC is inputted into the input of the switch controller 20. In the weight loss mode, the switch controller 20 directs the 12 volt DC through each first integrated circuit or circuit 44. Each first integrate circuit 44 or circuit converts 12 volt DC to a first output signal that is inputted to the corresponding LED 18 to cause the LED 18 to emit infrared radiation at a wave length of 635 nm that is sufficient to perforate one or more fat cells at the selected areas of the body 14 to drain the content within the one or more fat cells.

Alternatively, the user 12 may instead press the second push button 60 (or first button 58 a second time for the corresponding embodiment mentioned above) to place the switch controller 20 in the pain therapy mode. In the pain therapy mode, the switch controller 20 directs the 12 volt DC through each second integrated circuit or circuit 46. Each second integrate circuit or circuit 46 converts the 12 volt DC to a second output signal that is inputted to the corresponding LED 18 to cause the LED 18 to emit infrared radiation at a wave length of 850 nm to reduce pain and inflammation at the selected area of the body 14. Alternatively or additionally, the third push button 62 may be pushed by the user to place the switch in the visible light mode. In the visible light mode, the switch controller 20 directs the 12 volt DC through each third integrated circuit or circuit 47. Each third integrate circuit or circuit 47 converts the 12 volt DC to a third output signal that is inputted to the corresponding LED 18 to cause the LED 18 to emit infrared radiation at a wave length of 660 nm. Alternatively, the user 12 may operate the switch controller 20 to select the first, second, and/or third group of LEDs 18 which operate to emit radiation in a manner as previously mentioned. Alternatively, or in addition, the user may operate the switch controller 20 to enable operation of a combination of one two or three of the modes of operation at the same time or other time as previously mentioned. In step 108, the timer circuit 68 is activated to modulate the infrared light emitted by the LEDs 18 such that LEDs 18 turn off their light for one second for very four seconds. In step 110, the apparatus is then turn off to stop the supply of power to the apparatus 16.

Alternatively or in addition, the lights may be laser lights that are configured to emit light of the previously mentioned wavelengths that is sufficient to perforate one or more fat cells at the selected areas of the body to drain the content within the one or more fat cells, or to reduce pain and inflammation at the areas of the body as previously mentioned, or to provide visibility. In all other aspects, the apparatus that uses laser lights would be structured and function as that for the LEDs with the light emitting red and near infrared (NI) light to deliver the same reaction to the tissues of the user's body.

The apparatus has the following benefits. The apparatus used in the weight loss mode may cause fast and effective lipolysis of the fat cells for fat loss and detoxification, reduction of the appearance of cellulite, increased collagen formation for skin rejuvenation, and increased skin radiance and skin elasticity.

When the apparatus is used in the pain therapy mode, infrared therapy such as FAR-Infrared and LED phototherapy significantly reduces recovery time for wounds, bruises and injuries, increases oxygen and blood flow, improves circulation to the treated area and stimulates antioxidant enzyme release. Phototherapy in this spectrum also promotes muscle relaxation and generally facilitates pain reduction, stimulates fibroblast and collagen production, increases lymphatic drainage, and reduces swelling, inflammation and bruising—bringing relief to athletes, sufferers of arthritis, and those with chronic pain. When the apparatus is used in the visible light mode, the lights are visible and also emit radiation that helps to heal injuries, flatten skin, and smooth wrinkles. The apparatus also enables the user to conveniently select to have the wrap operate in the weight loss mode, the pain therapy mode and the visible light mode.

Although various embodiments of the disclosed apparatus and method for emitting light to a body of a user have been shown and described, modifications may occur to those skilled in the art upon reading the specification. The present application includes such modifications and is limited only by the scope of the claims.

What is claimed is:

1. An apparatus comprising:
a flexible wrap;
a plurality of lights arranged on the wrap;
wherein the wrap is configured to wrap around a body of a user such that the lights face selected areas of the body;
wherein the apparatus is configured to cause the lights to emit light at a first predetermined wavelength to the selected areas of the body that is sufficient to perforate one or more fat cells at the selected areas of the body to drain the content within the one or more fat cells when power is supplied to the lights; and
wherein the apparatus is configured to cause the lights to emit light at a second predetermined wavelength to the selected areas of the body at the same time as the lights emit light at the first predetermined wavelength when power is supplied to the lights.

2. The apparatus of claim 1 wherein the lights comprise LEDs, wherein the apparatus is configured to cause the LEDs to emit infrared radiation at the first predetermined wavelength to the selected areas of the body that is sufficient to perforate one or more fat cells at the selected areas of the body to drain the content within the one or more fat cells when power is supplied to the LEDs, wherein the apparatus is configured to cause the LEDs to emit infrared radiation at the second predetermined wavelength to the selected areas of the body at the same time as the LEDs emit infrared radiation at the first predetermined wavelength when power is supplied to the LEDs.

3. The apparatus of claim 1, wherein the first predetermined wavelength is generally between 630 nm and 635 nm.

4. The apparatus of claim 1 further including a switch, wherein the switch is configured to enable selection of either a first mode that causes the lights to emit light at the first predetermined wavelength to the selected areas of the body that is sufficient to perforate one or more fat cells at the selected areas of the body to drain the content within the one or more fat cells, or a second mode that causes the lights to emit light at the second predetermined wavelength to the selected areas of the body at the same time as the lights emit light at the first predetermined wavelength when power is supplied to the lights.

5. The apparatus of claim 4, wherein the apparatus is further configured to cause the lights to emit light at a third predetermined wavelength that is visible to the user when power is supplied to the light, wherein the switch is further configured to enable selection of a third mode that causes the lights to emit light at the third predetermined wavelength that is visible to the user at the same time as the lights emit light at the first predetermined wavelength and the second predetermined wavelength when power is supplied to the lights.

6. The apparatus of claim 1, wherein the second predetermined wavelength is between 830 nm and 880 nm.

7. The apparatus of claim 1, wherein the second predetermined wavelength is sufficient to increase circulation at the selected areas of the body.

8. The apparatus of claim 1, wherein the apparatus is further configured to cause each of the lights to also emit light at a third predetermined wavelength to the selected areas of the body at the same time as each of the lights emit light at the first predetermined wavelength and the second predetermined wavelength when power is supplied to the lights.

9. The apparatus of claim 8, wherein the third predetermined wavelength is generally 660 nm.

10. The apparatus of claim 1, wherein the array of lights consists of 30 columns and 10 rows.

11. The apparatus of claim 1, including a timer circuit, wherein the timer circuit causes the lights to emit light for one second every four seconds.

12. The apparatus of claim 1, wherein the ends of the wrap are configured to be detachably connected together.

13. A method comprising:
a) wrapping a wrap around a body of a user such that a plurality of lights on the wrap face selected areas of the body; and
b) supplying power to the wrap to cause the lights to emit light at a first predetermined wavelength to the selected areas of the body that is sufficient to perforate one or more fat cells at the selected areas of the body to drain the content within the one or more fat cells and simultaneously emit light at a second predetermined wavelength to the selected areas of the body to increase circulation at the selected areas of the body.

14. The method of claim 13, wherein wrapping the wrap around the body of the user such that the plurality of lights on the wrap face selected areas of the body further includes detachably connecting the ends of the wrap together.

15. The method of claim 13, wherein the lights comprise LEDs, wherein supplying power to the wrap causes the LEDs to emit infrared radiation at the first predetermined wavelength to the selected areas of the body that is sufficient to perforate one or more fat cells at the selected areas of the body to drain the content within the one or more fat cells and simultaneously emit infrared radiation at the second predetermined wavelength to the selected areas of the body to increase circulation at the selected areas of the body.

16. The method of claim 15 wherein the LEDs emit infrared light for one second every four seconds

17. The method of claim 13, wherein the first predetermined wavelength is generally between 630 nm and 635 nm.

18. The method of claim 13 further including a switch, wherein the switch is configured to enable selection of either a first mode that causes the lights to emit light at the first predetermined wavelength to the selected areas of the body that is sufficient to perforate one or more fat cells at the selected areas of the body to drain the content within the one or more fat cells, or a second mode that causes the lights to emit light at the second predetermined wavelength to the selected areas of the body at the same time as the lights emit light at the first predetermined wavelength when power is supplied to the lights, wherein the method further includes selecting the second mode.

19. The method of claim 13 further including a switch, wherein the switch is configured to enable selection of either:
a) a first mode that causes the lights to emit light at the first predetermined wavelength to the selected areas of the body that is sufficient to perforate one or more fat cells at the selected areas of the body to drain the content within the one or more fat cells;
b) a second mode that causes the lights to emit light at the second predetermined wavelength to the selected areas of the body at the same time as the lights emit light at the first predetermined wavelength when power is supplied to the lights; or
c) a third mode causes the lights to emit light at the third predetermined wavelength that is visible to the user at the same time as the lights emit light at the first predetermined wavelength and the second predetermined wavelength when power is supplied to the lights; and
wherein the method further includes selecting the third mode.

20. The method of claim 13, wherein the second predetermined wavelength is between 830 nm and 880 nm.
